(19) United States
(12) Patent Application Publication
(10) Pub. No.: US 2011/0189297 A1
(43) Pub. Date: Aug. 4, 2011

(54) STABLE SOLID ORAL DOSAGE CO-FORMULATIONS

(75) Inventors: Douglas N. Ludtke, Frederick, MD (US); Raymond E. Dagger, Mount Airy, MD (US)

(73) Assignee: Sequicia Pharmaceuticals, Gaithersburg, MD (US)

(21) Appl. No.: 13/119,394

(22) PCT Filed: Sep. 16, 2009
(86) PCT No.: PCT/US09/57183

§ 371 Date: Apr. 12, 2011

Related U.S. Application Data
(60) Provisional application No. 61/097,479, filed on Sep. 16, 2008.

(51) Int. Cl. A61K 9/14; A61K 31/27; A61P 31/18; A61K 31/4418

(52) U.S. Cl. 424/489; 514/479; 514/357

(57) ABSTRACT

Pharmaceutical compositions are provided that can act as boosters to improve the pharmacokinetics of drugs that undergo in vivo degradation by cytochrome P450 enzymes. Methods of inhibiting cytochrome P450 enzymes are provided that can be used for improving the treatment of diseases by preventing degradation of drugs or other molecules by cytochrome P450. Specifically, methods of inhibiting metabolic degradation of atazanavir sulphate for administering to a patient suffering from HIV infection are disclosed.

### FIELD OF THE INVENTION

Novel compositions and methods for improving the pharmacokinetics of drugs that undergo in vivo degradation by cytochrome P450 enzymes are provided.

### BACKGROUND OF THE INVENTION

Cytochrome P450s (P450) are a family of enzymes involved in the oxidative metabolism of both endogenous and exogenous compounds. P450 enzymes are widely distributed in the liver, intestines and other tissues (Krishna et al., Clinical Pharmacokinetics. 26:144-160, 1994). P450 enzymes catalyze the phase I reaction of drug metabolism, to generate metabolites for excretion. The classification of P450s is based on homology of the amino acid sequence (Slaughter et al The Annals of Pharmacotherapy 29:619-624, 1995). In mammals, there is over 55% homology of the amino acid sequence of CYP450 subfamilies. The differences in amino acid sequence constitute the basis for a classification of the superfamily of cytochrome P450 enzymes into families, subfamilies and isozymes.

Cytochrome P450 contains an iron cation and is a membrane bound enzyme that can carry out electron transfer and energy transfer. Cytochrome P450, when bound to carbon monoxide (CO), displays a maximum absorbance (peak) at 450 nm in the visible spectra, and is therefore called P450 (Omura et al., J. Biol. Chem. 239:2370, 1964).

Over 200 genes encoding cytochrome P450s have been identified, and are divided among over 30 gene families. These gene families are organized into subfamilies, which vary in regulation of gene expression and in amino acid sequence homology, substrate specificity, catalytic activity, and physiological role of the encoded enzymes. Representative P450 genes and substrates of the encoded enzymes are discussed below.

Listed below are examples of known substrates of members of various P450 subfamilies. See also the discussion in Klassen, ed., Casarett and Doull's Toxicology: The Basic Science of Poisons, McGraw-Hill, 1996, pp. 150 ff. Further information about cytochrome P450 substrates, can be found in Gonzales and other review articles cited above. Current information sources available via the Internet include the “Cytochrome P450 Homepage”, maintained by David Nelson, the “Cytochrome P450 Database”, provided by the Institute of Biomedical Chemistry & Center for Molecular Design, and the “Directory of P450-containing Systems”, provided by Kirill N. Degtyarenko and Peter Fabian.

CYP1A1: diethylstilbestrol, 2- and 4-hydroxyestradiol

CYP1A2: acetaminophen, phenacetin, acetanilide (analgesics), caffeine, clozapine (sedative), cyclobenzaprine (muscle relaxant), estradiol, imipramine (antidepressant), mexillitene (antiarrhythmic), naproxen (analgesic), riluzole, tacrine, theophylline (cardiac stimulant, bronchodilator, smooth muscle relaxant), warfarin. Cytochrome P450 family 2 (CYP2)

CYP2A6: coumarin, butadiene, nicotine

CYP2A13: nicotine

CYP2B1: phenobarbital, hexobarbital

CYP2C9: NSAIDs such as diclofenac, ibuprofen, and piroxicam; oral hypoglycemic agents such as tolbutamide and glipizide; angiotensin-2 blockers such as irbesartan, losartan, and valsartan; naproxen (analgesic); phenytoin (anticonvulsant, antiepileptic); sulfamethoxazole, tamoxifen (antineoplastic); torsemide; warfarin, flurbiprofen

CYP2C19: hexobarbital, mephobarbital, imipramine, clomipramine, citalopram, cycloguanil, the anti-epileptics phenytoin and diazepam, S-mephenytoin, diphenylhydantoin, lansoprazole, pantoprazole, omeprazole, pentamidine, propranolol, cyclophosphamide, progesterone

CYP2D6: antidepressants (imipramine, clomipramine, desimpramine), antipsychotics (haloperidol, perphenazine, risperidone, thioridazine), beta blockers (carvedilol, S-metoprolol, propafenone, timolol), amphetamine, codeine, dextromethorphan, fluoxetine, S-mexilletine, phenacetin, propranolol

CYP2E1: acetaminophen; chlorzoxazone (muscle relaxant), ethanol; caffeine, theophylline; dapsone, general anesthetics such as enflurane, halothane, and methoxyflurane; nitrosamines

CYP3A4: HIV Protease Inhibitors such as indinavir, ritonavir, lopinavir, amprenavir, tipranavir, darunavir, and saquinavir; HIV integrase inhibitors such as raltegravir, Hepatitis C virus (HCV) protease inhibitors, benzodiazepines such as alprazolam, diazepam, midazolam, and triazolam; immune modulators such as cyclosporine; antihistamines such as astemizole and chlorpheniramine; HMG CoA Reductase inhibitors such as atorvastatin, cerivastatin, lovastatin, and simvastatin; channel blockers such as diltiazem, felodipine, nifedipine, nisoldipine, nitrendipine, and verapamil; antibiotics such as clarithromycin, erythromycin, and rapamycin; various steroids including cortisol, testosterone, progesterone, estradiol, ethinylestradiol, hydrocortisone, prednisone, and prednisolone; acetominophen, aldrin, alfentanil, amiodarone, astemizole, benzphetamine, budesonide, carbemazepine, cyclophosphamide, ifosphamide, dapsone, digitoxin, quinidine (anti-arrhythmic), etoposide, flutamide, imipramine, lansoprazole, lidocaine, losartan, omeprazole, retinoic acid, FK506 (tacrolimus), tamoxifen, taxol and taxol analogs, e.g., taxotere, teniposide, terfenadine, buspirone, haloperidol (antipsychotic), methadone, sildenafil, trazodone, theophylline, toremifine, troleandomycin, warfarin, zatosetron, zonisamide.

CYP6A1: Fatty Acids.

The efficacy of a drug can be dramatically affected by its metabolism in the body. For drugs that are rapidly metabolized it can be difficult to maintain an effective therapeutic dose in the body, and the drug often must be given more frequently, in higher dose, and/or be administered in a sustained release formulation. Moreover, in the case of compounds for treating infectious disease, such as viral or bacterial infections, the inability to maintain an effective therapeutic dose can lead to the infectious agent becoming drug resistant. Many compounds that have strong biological efficacy and that would otherwise be potentially powerful therapeutics are rendered essentially useless by virtue of their short half-lives in vivo. A common pathway of metabolism for drugs containing lipophilic moieties is via oxidation by one or more cytochrome P450 enzymes. These enzymes metabolize a drug to a more polar derivative that is more readily excreted through the kidney or liver. First pass metabolism refers to the elimination of drugs via liver and intestinal CYP450 enzymes. First pass metabolism can lead to poor drug absorption from the GI tract due to extensive intestinal CYP450 metabolism, low plasma blood levels due to hepatic CYP450 metabolism, or both. Poor oral bioavailability due to CYP450 metabolism is a major reason for the failure of drugs candidates in clinical trials. In some instances, metabolic by-products of CYP450 enzymes are highly toxic and can result in severe side effects, cancer, and even death.

Some examples of the effects of drug metabolism by CYPs include:

Acetaminophen: Ethanol up-regulates CYP2E1, which metabolizes acetaminophen to a reactive quinone. This reactive quinone intermediate, when produced in sufficient amounts, causes liver damage and necrosis.

Sedatives: The sedative phenobarbital (PB) up-regulates several P450 genes, including those of the CYP2B and CYP3A subfamilies. Upregulation of these enzymes increases the metabolism and reduces the sedative effects of PB and the related sedative hexobarbital.

Antibiotics: The antibiotics rifampicin, rifampin, rifabutin, erythromycin, and related compounds are inducers of the CYP3A4 gene and are substrates of the enzyme product.

Anti-cancer agents: Taxol and taxotere are potent anti-cancer agents. Both drugs are extensively metabolized by CYP3A4 and have poor oral bioavailability. These drugs are only efficacious in parenteral formulations which, due to their poor solubility properties, are highly noxious to patients.

Nicotine: CYP2A6 and 2A13 convert nicotine, a non-toxic component of cigarette smoke, into NNK, a highly potent carcinogen and the cause of lung cancer from smoking.

Oral contraceptive/estrogen replacement therapy: Estrogens and estradiols are the active ingredients in oral contraceptives and in hormonal replacement therapies for post-menopausal women. Women who are also taking antibiotics such as rifampicin or erythromycin, or glucocorticoids such as dexamethasone, or who smoke, risk decreased efficacy of the estrogen/estradiol treatments due to increased metabolism of these compounds by up-regulated CYP3A4 and/or CYP1A2 enzymes.

Dextromethorphan: CYP2D6 metabolizes dextromethrophan to an inactive substance. Individuals who express high levels of CYP2D6 (so-called rapid metabolizers) do not receive therapeutic benefits from dextromethorphan due to extensive first-pass metabolism and rapid systemic clearance.

Protease Inhibitors: All protease inhibitors and non-nucleoside reverse transcriptase inhibitors currently indicated for use in treatment of HIV or HCV are typically good substrates of cytochrome P450 enzymes; in particular, they are metabolized by CYP3A4 enzymes (see e.g. Sahai, AIDS 10 Suppl 1:S21-5, 1996) with possible participation by CYP2D6 enzymes (Kumar et al., J. Pharmacol. Exp. Ther. 277(1):423-31, 1996). Although protease inhibitors are reported to be inhibitors of CYP3A4, some non-nucleoside reverse transcriptase inhibitors, such as nevirapine and efavirenz, are inducers of CYP3A4 (see e.g. Murphy et al., Expert Opin Invest Drugs 5/9: 1183-99, 1996).

Human CYP450 isozymes are widely distributed among tissues and organs (Zhang et al., Drug Metabolism and Disposition. 27:804-809, 1999). With the exception of CYP1A1 and CYP2A13, most human CYP450 isozymes are located in the liver, but are expressed at different levels (Waziers J. Pharmacol. Exp. Ther. 253:387, 1990). A solution to the problem of drug degradation and first-pass metabolism is to control the rate of drug metabolism. When the rates of absorption and metabolism reach a steady state, a maintenance dose can be delivered to achieve a desired drug concentration that is required for drug efficacy. Certain natural products have been shown to increase bioavailability of a drug. For example, the effect of grapefruit juice on drug pharmacokinetics is well known. See Edgar et al., Eur. J. Clin. Pharmacol. 42:313, (1992); Lee et al., Clin. Pharmacol. Ther. 59:62, (1996); Kane et al., Mayo Clinic Proc. 75:933, (2000). This effect of grapefruit juice is due to the presence of natural P450-inhibiting components. Other compounds also have been used for inhibition of P450. For example, the HIV-1 protease inhibitor Ritonavir® is now more commonly prescribed for use in combination with other, more effective HIV protease inhibitors because of its ability to “boost” those other compounds by inhibiting P450-mediated degradation.

Current methods of inhibiting cytochrome P450 enzymes are not wholly satisfactory because of toxicity issues, high cost, and other factors. For example, using Ritonavir® [(2S,3S,5S)-5-(N—(N—((N-methyl-N-((2-isopropyl-4-thiazolyl)methyl)amino)carbonyl)-L-valinyl)amino-2-(N-((5-thiazolyl)methoxy-carbonyl)-amino)-amino-1,6-diphenyl-3hydroxyhexane] to inhibit cytochrome P450 is not desirable in disorders other than HIV infection. It is apparent, therefore, that new and improved methods of inhibiting cytochrome P450 enzymes are greatly to be desired. In particular, methods where an inhibitor can be co-administered with another biologically active compound that is metabolized by cytochrome P450 enzymes are highly desirable.

### SUMMARY OF THE INVENTION

It is therefore an object of the present invention to provide new formulations of cytochrome p450 inhibitors that provide enhanced bioavailability of the inhibitors.

It is also an object of the present invention to provide methods of inhibiting cytochrome p450 by administering a formulation of a cytochrome p450 inhibitor with enhanced bioavailability.

In accomplishing these objects, there has been provided, in accordance with one aspect of the present invention, a pharmaceutical composition containing an amorphous dispersion of an effective amount of a cytochrome p450 inhibitor and a water soluble polymer, where the amorphous dispersion has a glass transition temperature (Tg) of about 75° C. or greater and inhibits plasticization upon exposure to gastric fluid, and a disintegrant. The composition may also contain an active pharmaceutical agent, where the active pharmaceutical agent is a substrate for human cytochrome p450, where the active pharmaceutical agent may be, but need not be, contained in the amorphous dispersion.

In one embodiment, the cytochrome p450 inhibitor has the formula:

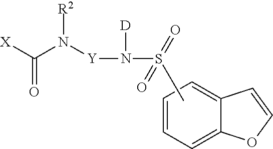

where:

X is C₁-C₁₂ alkyl, cycloalkyl, aryl, aralkyl, heteroaryl, or heteroaralkyl, optionally substituted with one or more substituents selected from the group consisting of halo, OR, ROH, R-halo, CN, COₙR, CON(R)₂, SOₙN(R)₂, SR, SOₙR, N(R)₂, N(R)COₙR, NRS(O)ₙR, oxo, and ═N—OR; Y is —(CG₁G₂)ₘ-, where m is 2-6 and where G₁ and G₂ are the same or different and where each G₁ and G₂ independently is selected from the group consisting of a bond, H, OR, optionally substituted alkyl, optionally substituted aryl, optionally substituted cycloalkyl, optionally substituted cycloalkylalkyl, optionally substituted aralkyl, optionally substituted heteroaryl, and optionally substituted heteroaralkyl, where each optional substitution independently is selected from the group consisting of alkyl , halo, cyano, CF₃, OR, C₃-C₇ cycloalkyl, C₅-C₇ cycloalkenyl, R6, OR2, SR2, N(R2)₂, OR3, SR3, NR2R3, OR6, SR6, and NR2R6; D is selected from the group consisting of hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, O-cycloalkyl, O-cycloalkylalkyl, O-heterocycloalkyl, O-heterocycloalkylalkyl, O-aralkyl, N(R2)-alkyl, N(R2)-cycloalkyl, N(R2)-cycloalkylalkyl, N(R2)-heterocycloalkyl, N(R2)-heterocycloalkylalkyl, N(R2)-heteroaralkyl, N(R2)-aralkyl, where D optionally is substituted by alkyl, halo, nitro, cyano, O-alkyl, or S-alkyl; where R is H, alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl; where each R2 is independently selected from the group consisting of H, C₁-C₁₂ alkyl, C₃-C₈ cycloalkyl, aryl, aralkyl, heteroaryl, heteroaralkyl, and heterocycloalkyl each further optionally substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R); or each R2 is independently selected from the group consisting of C₁-C₆ alkyl; substituted by aryl or heteroaryl; which groups optionally are substituted with one or more substituents selected from the group consisting of halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR; R3 is C₂-C₆ alkenyl, C₂-C₆ alkynyl, C₃-C₈ cycloalkyl, C₅-C₈ cycloalkenyl, or heterocyclo; which groups optionally are substituted with one or more substituents selected from the group consisting of halo, OR2, R2-OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, oxo, ═N—OR2, ═N—N(R2)₂, ═NR2, ═NNRC(O)N(R2)₂, ═NNR2C(O)ₙR2, ═NNR2S(O)ₙN(R2)₂, and ═NNR2S(O)ₙ(R2); R6 is aryl or heteroaryl, where the aryl or heteroaryl optionally are substituted with one or more groups selected from the group consisting of aryl, heteroaryl, R2, R3, halo, OR2, R2OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, OC(O)R2, OC(S)R2, OC(O)N(R2)₂, and OC(S)N(R2)₂; and where =1-2.

In a specific embodiment, X is C₁-C₁₂ alkyl, m is 3, one G₁ is alkoxy, and a second G₁ is optionally substituted aralkyl, and D is alkyl. In another embodiment, the cytochrome p450 inhibitor has the formula:

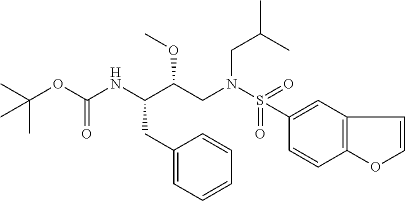

In these compositions, the water soluble polymer may be is selected from the group consisting of polyvinyl acetate phthalate, hydroxypropylmethyl-cellulose acetate succinate, cellulose acetate phthalate, methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate, cellulose propionate phthalate, cellulose acetate maleate, cellulose acetate trimellitate, cellulose acetate butyrate, cellulose acetate propionate, methacrylic acid/methacrylate polymer, methacrylic acid-methyl methacrylate copolymer, ethyl methacrylate-methylmethacrylate-chlorotrimethylammonium ethyl methacrylate copolymer, shellac, copal collophorium, carageenan, alginic acid and salts thereof, karaya gum, acacia gum, tragacanth gum, locust bean gum, guar gum, sodium carboxymethyl cellulose, methyl cellulose, and combinations thereof. In a specific embodiment, the water soluble polymer is a polymethacrylate, for example Eudragit L100-55 or Eudragit L100. The amorphous dispersion may be a spray-dried dispersion, and may, for example, have an average particle diameter of <100 micron. In a particular embodiment, the dispersion has a glass transition temperature (Tg) between about 100° C. and about 125° C.

When an active pharmaceutical agent is present, it may be selected from the group consisting of Cyclosporine, Tacrolimus (FK506), Sirolimus (rapamycin), Indinavir, Ritonavir, Saquinavir, Felodipine, Isradipine, Nicardipine, Nisoldipine, Nimodipine, Nitrendipine, Nifedipine, Verapamil, Etoposide, Tamoxifen, Vinblastine, Vincristine, Taxol, Atorvastatin, Fluvastatin, Lovastatin, Pravastatin, Simvastatin, Terfenadine, Loratadine, Astemizole, Alfentanil, Carbamazepine, Azithromycin, Clarithromycin, Erythromycin, Itraconazole, Rifabutin, Lidocaine, Cisapride, Sertraline, Pimozide, Triazolam, Anastrazole, Busulfan, Corticosteroids (dexamethasone, methylprednisone and prednisone), Cyclophosphamide, Cytarabine, Docetaxel, Doxorubicin, Erlotinib, Exemestane, Gefitinib, Idarubicin, Ifosphamide, Imatinib mesylate, Irinotecan, Ketoconazole, Letrozole, Paclitaxel, Teniposide, Tretinoin, Vinorelbine,telithromycin: quinidine; alprazolam, diazepam, midazolam, nelfinavir, chlorpheniramine, amlodipine, diltiazem, lercanidipine, cerivastatin, estradiol, hydrocortisone, progesterone, testosterone, alfentanyl, aripiprazole, buspirone, cafergot, caffeine, cilostazol, codeine, dapsone, dextromethorphan, docetaxel, domperidone, eplerenone, fentanyl, finasteride, Gleevec®, haloperidol, irinotecan, Levo-Alpha Acetyl Methadol (LAAM), methadone, nateglinide, odanestron, propranolol, quinine, salmetrol, sildenafil, terfenadine, trazodone, vincristine, zaleplon, zolpidem., ixabepilone, Agenerase, Aptivus, Crixivan, Invirase, Lexiva, Prezista, Reyataz, Viracept, Elvitegravir, Selzentry, Vicriviroc, Telaprevir, Telithromycin, tandospirone, buspirone, pharmaceutically acceptable salts, crystalline forms, non-crystalline forms and polymorphs thereof.

In these compositions, the water soluble polymer may be selected so as to increase the solubility of the cytochrome p450 inhibitor at a pH of greater than 5.5. In a specific embodiment, the cytochrome p450 inhibitor and a water soluble polymer may be present in a ratio ranging from 1.6:0.4 to 0.4:1.6.

The composition may be in an oral dosage form, such as a powder, granules, tablet, pill or capsule. The composition may be free of lipid or oil solvent.

In specific embodiments, the cytochrome p450 inhibitor and active pharmaceutical agent may each be present in an amount ranging from about 0.1 wt. % to about 80 wt. %.

In other embodiments, the disintegrant is selected from the group consisting of microcrystalline cellulose, sodium starch glycolate, cross-linked carboxymethylcellulose and its sodium salt, cross-linked polyvinylpyrrolidone, pregelatinised starch, sodium carboxymethyl cellulose, calcium carboxymethyl cellulose, low-substituted hydroxypropyl cellulose, alginates or its salts and mixtures thereof. The composition may also contain a diluent, for example, lactose, dextrose, sucrose, fructose, maltose, powdered cellulose, microcrystalline cellulose, mannitol, erythritol, sorbitol, xylitol, lactitol, dicalcium phosphate, tribasic calcium phosphate, calcium sulphate, calcium carbonate and/or mixtures thereof.

The composition may also contain at least one binder, for example, corn starch, pregelatinised starch, polyvinylpyrrolidone, hydroxypropyl cellulose, hydroxypropyl methylcellulose, carboxyvinyl polymers, acrylates and/or mixtures thereof, and may also contain at least one lubricant, for example talc, magnesium stearate, zinc stearate, calcium stearate, sodium stearyl fumarate, stearic acid and/or mixtures thereof.

The composition may also contain least one glidant, for example, talc, colloidal silicon dioxide and mixtures thereof.

In a specific embodiment, the active pharmaceutical agent may be atazanavir, such as atazanavir sulfate. The atazanavir sulfate may be formulated as a powder in combination with an excipient mixture containing, for example, crospovidone, lactose monohydrate and magnesium stearate.

In accordance with another object of the invention, there has been provided a method of inhibiting cytochrome p450 in a subject, by administering to the subject an effective amount of a composition as described above.

In accordance with another object of the invention, there has been provided a method of treating a patient suffering from HIV infection, by administering to the patient a composition as described above, containing an active pharmaceutical agent that is an HIV inhibitor, such as an HIV protease inhibitor.

In accordance with another object of the invention, there has been provided a water-dispersible pharmaceutical dosage formulation suitable for oral administration comprising (i) an effective amount of a spray-dried amorphous dispersion of a compound having the formula:

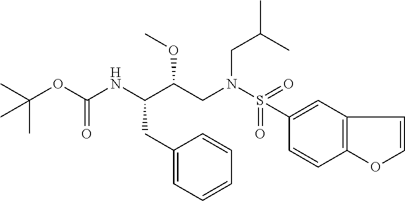

and a methacrylic acid-acrylic acid ethyl ester copolymer, where the dispersion has a glass transition temperature (Tg) in excess of 75° C., in combination with (ii) a drug selected from the group consisting of Cyclosporine, Tacrolimus (FK506), Sirolimus (rapamycin), Indinavir, Ritonavir, Saquinavir, Felodipine, Isradipine, Nicardipine, Nisoldipine, Nimodipine, Nitrendipine, Nifedipine, Verapamil, Etoposide, Tamoxifen, Vinblastine, Vincristine, Taxol, Atorvastatin, Fluvastatin, Lovastatin, Pravastatin, Simvastatin, Terfenadine, Loratadine, Astemizole, Alfentanil, Carbamazepine, Azithromycin, Clarithromycin, Erythromycin, Itraconazole, Rifabutin, Lidocaine, Cisapride, Sertraline, Pimozide, Triazolam, Anastrazole, Busulfan, Corticosteroids (dexamethasone, methylprednisone and prednisone), Cyclophosphamide, Cytarabine, Docetaxel, Doxorubicin, Erlotinib, Exemestane, Gefitinib, Idarubicin, Ifosphamide, Imatinib mesylate, Irinotecan, Ketoconazole, Letrozole, Paclitaxel, Teniposide, Tretinoin, Vinorelbine,telithromycin: quinidine; alprazolam, diazepam, midazolam, nelfinavir, chlorpheniramine, amlodipine, diltiazem, lercanidipine, cerivastatin, estradiol, hydrocortisone, progesterone, testosterone, alfentanyl, aripiprazole, buspirone, cafergot, caffeine, cilostazol, codeine, dapsone, dextromethorphan, docetaxel, domperidone, eplerenone, fentanyl, finasteride, gleevec, haloperidol, irinotecan, Levo-Alpha Acetyl Methadol (LAAM), methadone, nateglinide, odanestron, propranolol, quinine, salmetrol, sildenafil, terfenadine, trazodone, vincristine, zaleplon, zolpidem., ixabepilone, Agenerase, Aptivus, Crixivan, Invirase, Lexiva, Prezista, Reyataz,Viracept, Elvitegravir, Selzentry, Vicriviroc, Telaprevir, Telithromycin, tandospirone, buspirone, pharmaceutically acceptable salts, crystalline forms, non-crystalline forms and polymorphs thereof.

In accordance with another object of the invention, there has been provided a solid gelatin capsule suitable for oral administration containing:

(i) an effective amount of a spray-dried amorphous dispersion of a compound having the formula:

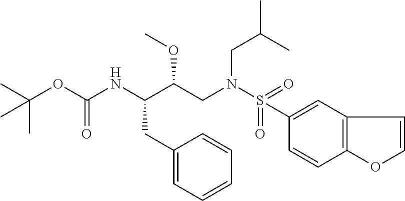

and a methacrylic acid-acrylic acid ethyl ester copolymer in the ratio 1:1, where the dispersion has a glass transition temperature (Tg) in excess of 75° C.;

(ii) atazanavir sulphate in a powder form where the powder comprises crospovidone, lactose monohydrate and magnesium stearate.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows the dissolution of compound II (lower curve) and atazanavir (upper curve) in a formulation of the invention

FIG. 2 shows the mean (Std Dev) plasma concentrations of atazanavir in Dogs (n=4) dosed orally with dry powder(s) in capsules either of 20 mg atazanavir/kg alone, or with atazanavir plus 10 mg/kg Compound II from Compound II:EL100

FIG. 3 shows the mean (Std Dev) plasma concentrations of Compound II in Dogs (n=4) dosed orally with 10 mg Compound II/kg from Compound II:EL100 either alone or in combination with 20 mg/kg Atazanavir, as dry powder in capsules

### DETAILED DESCRIPTION OF THE INVENTION

Novel pharmaceutical compositions are provided that permit convenient and palatable dosage of cytochrome p450 inhibitors in a form that provides excellent pharmacokinetics upon oral administration. Specifically, the compositions permit efficient oral administration of hydrophobic p450 inhibitors, either alone or in combination with a second pharmaceutical compound that is degraded in vivo by cytochrome p450. In this manner the p450 inhibitors act as pharmacokinetic enhancers to improve the effectiveness of the second pharmaceutical compound. Moreover, the novel compositions are surprisingly effective at permitting efficient dosing of hydrophobic p450 inhibitors that otherwise have low bioavailability.

More specifically, the compositions of the invention comprise an amorphous dispersion of an effective amount of a cytochrome p450 inhibitor and a water soluble polymer, together with a disintegrant. The amorphous dispersion has a glass transition temperature (Tg) of about 75° C. or greater and inhibits plasticization upon exposure to gastric fluid. This high Tg means that the amorphous nature of the composition, and resulting bioavailability, is preserved during storage. In compositions in which a second pharmaceutical agent is present, that second agent may be added separately to the amorphous dispersion, or may be present as a part of the amorphous dispersion, as desired. More specifically, for second pharmaceutical agents that already have acceptable bioavailability it typically is not necessary to incorporate the second agent into the amorphous dispersion, while for second agents where bioavailability may be an issue, it may be advantageous to incorporate the agent into the amorphous dispersion.

Cytochrome p450 Inhibitors

The compositions described herein advantageously may be used to formulate p450 inhibitors that have proven difficult to formulate and deliver by conventional methods. In particular, the compositions are useful for formulating p450 inhibitors that are very hydrophobic and/or that suffer from low bioavailability when formulated and administered using conventional methods. In a particular embodiment, cytochrome p450 inhibitor has the formula I:

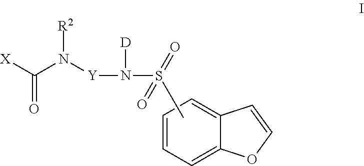

where X is C₁-C₁₂ alkyl, cycloalkyl, aryl, aralkyl, heteroaryl, or heteroaralkyl, optionally substituted with one or more substituents selected from the group consisting of halo, OR, ROH, R-halo, CN, COₙR, CON(R)₂, SOₙN(R)₂, SR, SOₙR, N(R)₂, N(R)COₙR, NRS(O)ₙR, oxo, and ═N—OR. Y is —(CG₁G₂)ₘ-, where m is 2-6 and where G₁ and G₂ are the same or different and where each G₁ and G₂ independently is selected from the group consisting of a bond, H, OR, optionally substituted alkyl, optionally substituted aryl, optionally substituted cycloalkyl, optionally substituted cycloalkylalkyl, optionally substituted aralkyl, optionally substituted heteroaryl, and optionally substituted heteroaralkyl, and where each optional substitution independently is selected from the group consisting of alkyl , halo, cyano, CF₃, OR, C₃-C₇ cycloalkyl, C₅-C₇ cycloalkenyl, R6, OR2, SR2, N(R2)₂, OR3, SR3, NR2R3, OR6, SR6, and NR2R6,

D is hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, O-cycloalkyl, O-cycloalkylalkyl, O-heterocycloalkyl, O-heterocycloalkylalkyl, O-heteroaralkyl O-aralkyl, N(R2)-alkyl, N(R2)-cycloalkyl, N(R2)-cycloalkylalkyl, N(R2)-heterocycloalkyl, N(R2)-heterocycloalkylalkyl, N(R2)-heteroaralkyl, or N(R2)-aralkyl, where D optionally is substituted by alkyl, halo, nitro, cyano, O-alkyl, or S-alkyl;

R is H, alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;

each R2 is independently selected from the group consisting of H, C₁-C₁₂ alkyl, C₃-C₈ cycloalkyl, aryl, aralkyl, heteroaryl, heteroaralkyl, and heterocycloalkyl each further optionally substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);

or each R2 is independently selected from the group consisting of C₁-C₆ alkyl; substituted by aryl or heteroaryl; which groups optionally are substituted with one or more substituents selected from the group consisting of halo, OR, ROH, R-halo, NO₂, CN, COₙR, CON(R)₂, C(S)R, C(S)N(R)₂, SOₙN(R)₂, SR, SO_{A}R, N(R)₂, N(R)COₙR, NRS(O)ₙR, NRC[═N(R)]N(R)₂, N(R)N(R)COₙR, NRPOₙN(R)₂, NRPOₙOR;

R3 is C₂-C₆ alkenyl, C₂-C₆ alkynyl, C₃-C₈ cycloalkyl, C₅-C₈ cycloalkenyl, or heterocyclo; which groups optionally are substituted with one or more substituents selected from the group consisting of halo, OR2, R2-OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, oxo, ═N—OR2, ═N—N(R2)₂, ═NR2, ═NNRC(O)N(R2)₂, ═NNR2C(O)ₙR2, ═NNR2S(O)ₙN(R2)₂, and ═NNR2S(O)ₙ(R2);

R6 is aryl or heteroaryl, wherein said aryl or heteroaryl optionally are substituted with one or more groups selected from the group consisting of aryl, heteroaryl, R2, R3, halo, OR2, R2OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, OC(O)R2, OC(S)R2, OC(O)N(R2)₂, and OC(S)N(R2)₂; and

where n=1-2.

Advantageously, X is C₁-C₁₂ alkyl, m is 3, one G₁ is alkoxy, and a second G₁ is optionally substituted aralkyl, and D is alkyl.

In a specific embodiment, the p450 inhibitor has the structure II:

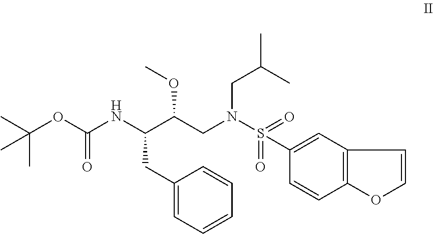

This molecule (“the compound of Formula II” or “Compound II”) possesses no inherent anti-retroviral activity and functions as a pure pharmacokinetic enhancer:

Methods of Making Formulations with High Bioavailability

The amorphous dispersion of the p450 inhibitor may be prepared using methods that are known in the art. Advantageously, however, the dispersion is prepared by spray-drying. The phrase “spray-dried amorphous dispersion” defines a system in a solid state comprising at least two components, where one component is dispersed more or less evenly throughout the other component or components.

For preparation by spray-drying, the p450 inhibitor is mixed with a water soluble polymer in a suitable solvent. Suitable solvents include organic solvents such as dichloromethane and the like. The ratio of inhibitor to polymer may be varied as required, but advantageously, an approximately 50% (w/w) ration may be used, although ratios from 10% to 80%, advantageously 20%-70,% or 30%-60% may be used as appropriate. The solution is then spray dried using methods that are known in the art and the resulting amorphous dispersion is collected.

The skilled artisan will recognize that a wide variety of water soluble polymers may be used in the compositions of the present invention. For example, polymers that may be used include polyvinyl acetate phthalate, hydroxypropylmethyl-cellulose acetate succinate, cellulose acetate phthalate, methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate, cellulose propionate phthalate, cellulose acetate maleate, cellulose acetate trimellitate, cellulose acetate butyrate, cellulose acetate propionate, methacrylic acid/methacrylate polymer, methacrylic acid-methyl methacrylate copolymer, ethyl methacrylate-methylmethacrylate-chlorotrimethylammonium ethyl methacrylate copolymer, shellac, copal collophorium, carageenan, alginic acid and salts thereof, karaya gum, acacia gum, tragacanth gum, locust bean gum, guar gum, sodium carboxymethyl cellulose, methyl cellulose, and combinations of these polymers Advantageously, the polymer is a methacrylic acid copolymer(polymethacrylate). Suitable polymethacrylate polymers are available commercials as, for example, Eudragit L100-55 and Eudragit L100 (Evonik Industries, Essen, Germany).

In a typical preparation, a spray-dried amorphous dispersion (SDD) of the compound of formula II (above) with EUDRAGIT L-100 (EL-100) is prepared. Laboratory scale spray dryers, SD42 and SD44 (BÜCHI, model B-290 Advanced) may be used for spray drying. The compound of formula II is present in this SDD in a stabilized amorphous form, which enhances its absorption in biological systems. One specific foimulation that may be used for the oral dosage form is Compound:EL 100, 1:1 SDD. This preparation presents the p450 inhibitor as a 50% (w/w) dispersion of EL100.

This dispersion has a high glass transition temperature (T_{g}=125° C.), indicative of excellent physical stability. Methods for determining Tg values of the organic polymers are well known in the art and are described, for example, in “Introduction to Physical Polymer Science”, 2^{nd}, L. H. Sperling (editor), John Wiley & Sons, 1992.

Once prepared, the SDD may be mixed with a disintegrant (dispersant) to prepare the final dosage form. Suitable disintegrants typically are materials that expand on exposure to aqueous environments and include microcrystalline cellulose, sodium starch glycolate, cross-linked carboxymethylcellulose and its sodium salt, cross-linked polyvinylpyrrolidone, pregelatinised starch, sodium carboxymethyl cellulose, calcium carboxymethyl cellulose, low-substituted hydroxypropyl cellulose, alginates or salts and mixtures thereof. Advantageously, the disintegrant is microcrystalline cellulose. The amount of disintegrant that is used can be varied to achieve disintegration of the dosage form, and methods of determining the optimal properties by varying the quantity of disintegrant are well known in the art. When mixed with a disintegrant such as microcrystalline cellulose the SDD as described above does not plasticize on exposure to aqueous systems but, as desired, remains in suspension as finely divided particles.

As a solid foimulation for oral administration, the composition of the present invention can be in the form of powders, granules, tablets, pills and capsules. In these cases, the active agents of the instant formulation can be further mixed with conventional additives, fillers, diluents, lubricants, preservatives, glidants, anti-oxidants, binders, thickening agents, buffers, sweeteners, flavoring agents, perfuming agents and the like. Examples of suitable lubricants include stearic acid, magnesium stearate, glyceryl behenate, talc, mineral oil (in PEG), and combinations comprising one or more of the foregoing lubricants. Examples of suitable binders include water-soluble polymer, such as modified starch, gelatin, polyvinylpyrrolidone, polyvinyl alcohol, and combinations comprising one or more of the foregoing lubricants. An example of a glidant is silicon dioxide (AEROSIL, Degussa). Suitable fillers include insoluble materials such as silicon dioxide, titanium dioxide, talc, alumina, starch, kaolin, polacrilin potassium, powdered cellulose, and microcrystalline cellulose, and combinations comprising one or more of the foregoing fillers.

By “oral dosage form” is meant to include a unit dosage form prescribed or intended for oral administration. An oral dosage form may or may not comprise a plurality of subunits such as, for example, microcapsules or microtablets, packaged for administration in a single dose. The term “dosage form” denotes a form of a formulation that contains an amount sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics such as solubility, and with the characteristics of the swellable matrix such as its permeability, and the relative amounts of the drug and polymer. The dosage form can be prepared by various conventional mixing, comminution and fabrication techniques readily apparent to those skilled in the chemistry of drug formulations.

Certain oral dosage forms described herein may be “coated”. The coating can be a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating. Note that a non-functional coating can have some impact on the release of the active agent due to the initial dissolution, hydration or perforation of the coating but would not be considered to be a significant deviation from the non-coated composition.

“Enteric coated” formulations, which protect the stomach against any irritant effects of the active agent(s), are also possible within the scope of this invention. Such formulations can be coated with a composition that is non-toxic and includes a pharmaceutically acceptable enteric polymer which is predominantly soluble in the intestinal fluid while being substantially insoluble in the low pH of the gastric juices. Examples include polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L, which is an anionic copolymer based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups. Further examples include non-pH dependent polymers like carageenan, alginic acid and salts thereof, karaya gum, acacia gum, trgacanth gum, locust bean gum, guar gum, sodium carboxymethyl cellulose, and methyl cellulose x. The methacrylic acid/acrylic acid ethyl ester copolymer solid substance sold under the trade designation “EUDRAGIT L-100” is particularly suitable for the present invention.

Combinations with a Second Pharmaceutical Compound

As described above, the formulations of the p450 inhibitor may also contain a second pharmaceutical compound, advantageously a compound that is a substrate for cytochrome p450. As such, the p450 inhibitor acts to prevent degradation of the second compound, thereby “boosting” the pharmacokinetic properties of that compound. When the second compound is hydrophobic and/or otherwise has poor aqueous solubility such that it has poor bioavailability, the second compound may advantageously be included in the SDD preparation together with the p450 inhibitor; otherwise it may be added separately after preparation of the SDD. The second compound may be admixed with the SDD preparation and disintegrant as a pure compound, or may be preformulated in a suitable manner prior to such mixing. For example, the second compound may be formulated into granules that permit extended or immediate release of the compound as desired.

Exemplary compounds that may be incorporated into formulations together with the SDD desribed above include, but are not limited to: Cyclosporine, Tacrolimus (FK506), Sirolimus (rapamycin), Indinavir, Ritonavir, Saquinavir, Felodipine, Isradipine, Nicardipine, Nisoldipine, Nimodipine, Nitrendipine, Nifedipine, Verapamil, Etoposide, Tamoxifen, Vinblastine, Vincristine, Taxol, Atorvastatin, Fluvastatin, Lovastatin, Pravastatin, Simvastatin, Terfenadine, Loratadine, Astemizole, Alfentanil, Carbamazepine, Azithromycin, Clarithromycin, Erythromycin, Itraconazole, Rifabutin, Lidocaine, Cisapride, Sertraline, Pimozide, Triazolam, Anastrazole, Busulfan, Corticosteroids (dexamethasone, methylprednisone and prednisone), Cyclophosphamide, Cytarabine, Docetaxel, Doxorubicin, Erlotinib, Exemestane, Gefitinib, Idarubicin, Ifosphamide, Imatinib mesylate, Irinotecan, Ketoconazole, Letrozole, Paclitaxel, Teniposide, Tretinoin, Vinorelbine,telithromycin: quinidine; alprazolam, diazepam, midazolam, nelfinavir, chlorpheniramine, amlodipine, diltiazem, lercanidipine, cerivastatin, estradiol, hydrocortisone, progesterone, testosterone, alfentanyl, aripiprazole, buspirone, cafergot, caffeine, cilostazol, codeine, dapsone, dextromethorphan, docetaxel, domperidone, eplerenone, fentanyl, finasteride, gleevec, haloperidol, irinotecan, Levo-Alpha Acetyl Methadol, methadone, nateglinide, odanestron, propranolol, quinine, salmetrol, sildenafil, terfenadine, trazodone, vincristine, zaleplon, zolpidem., ixabepilone, Agenerase, Aptivus, Crixivan, Invirase, Lexiva, Prezista, Reyataz, Viracept, Elvitegravir, Seizentry, Vicriviroc, Telaprevir, Telithromycin, tandospirone, and buspirone, pharmaceutically acceptable salts, crystalline foiius, non-crystalline forms and polymorphs thereof.

The active agents of the instant invention can be administered in the form of “pharmaceutically acceptable salts” derived from inorganic or organic acids, wherein the parent compound is modified by making non-toxic acid or base salts addition thereof, or as pharmaceutically acceptable solvates (including hydrates), crystalline and non-crystalline forms as well as various polymorphs thereof. Included among such acid salts, for example, are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate. Other pharmaceutically acceptable salts include salts with an inorganic base, organic base, inorganic acid, organic acid, or basic or acidic amino acid. Inorganic bases which form the pharmaceutically acceptable salts include alkali metals such as sodium or potassium, alkali earth metals such as calcium and magnesium, aluminum, and ammonia. Organic bases which form pharmaceutically acceptable salts include trimethylamine, triethylamine, pyridine, picoline, ethanolamine, diethanolamine, triethanolamine, dicyclohexylamine. Inorganic acids which form the pharmaceutically acceptable salts include hydrochloric acid, hydroboric acid, nitric acid, sulfuric acid, and phosphoric acid. Organic acids appropriate to form the salt include formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. Basic amino acids to form the salt include arginine, lysine and ornithine. Acidic amino acids to form the salt include aspartic acid and glutamic acid.

An exemplary formulation is provided in the examples below that contains the compound of Formula II and the HIV-1 protease inhibitor, Atazanavir (Bristol-Myers Squibb Co.). Atazanavir, trade name Reyataz® (formerly known as BMS-232632) is an antiretroviral drug of the protease inhibitor class used to treat infection HIV. Atazanavir is extensively metabolized in humans, primarily by the liver. In vitro studies using human liver microsomes suggested that Atazanavir is metabolized by CYP3A. Reyataz® capsules contain Atazanavir as Atazanavir sulfate along with crospovidone, lactose monohydrate and magnesium stearate. (Azapeptide HIV protease inhibitor. Prepn: A. Fässler et al., WO 9740029; eidem, U.S. Pat. No. 5,849,911 (1997, 1998 both to Novartis); G. Bold et al, J. Med. Chem. 41, 3387 (1998); of bisulfate salt: J. Singh et al, WO 9936404; eidem, U.S. Pat. No. 6,087,383 (1999, 2000 both to Bristol-Myers Squibb); Z. Xu et al., Org. Process Res. Dev. 6, 323 (2002). Comparative anti-HIV activity: B. S. Robinson et al., Antimicrob. Agents Chemother. 44, 2093 (2000). HPLC determination in plasma: E. Cateau et al., J. Pharm. Biomed. Anal. 39, 791 (2005). Clinical evaluation in HIV: I. Sanne et al, J. Acquired Immune Defic. Synd. 32, 18 (2003). Review of pharmacology and clinical efficacy in HIV: C. Le Tiec et al., Clin. Pharmacokinet. 44, 1035-1050 (2005); T. S. Harrison, L. J. Scott, Drugs 65, 2309-2336 (2005).)

Atazanavir sulfate has the following structural formula:

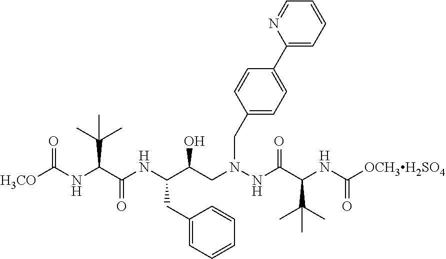

As described below, coformulation of Atazanavir with the compound of Formula II provided a significantly improved pharmacokinetic profile for Atazanavir.

Methods of Treatment Using Oral Dosage Forms

Dosages of the compounds are dependent on age, body weight, general health conditions, sex, diet, dose interval, administration routes, excretion rate, combinations of drugs and conditions of the diseases treated, while taking these and other necessary factors into consideration. The amounts of the two active agents (the compound of Formula II and Atazanavir) that can be combined with the carrier materials to produce a single dosage form will vary depending upon the host treated and the particular mode of administration. A typical preparation will contain from about 5% to about 95% of each active agent (w/w). Preferably, such preparations contain from about 20% to about 80% of each active agent. The desired unit dose of the composition of this technology is administered once or multiple times daily.

The term “subject” as employed herein refers to a mammal, and, more particularly to a human.

It should also be understood that the various embodiments of the present invention are not mutually exclusive, but may be implemented in various combinations. The various compositions, methods and embodiments shown herein are not limiting of the invention, and any obvious modifications will be apparent to one skilled in the art.

Unless otherwise defined herein, scientific and technical terms used in connection with the present invention shall have the meanings that are commonly understood by those of ordinary skill in the art. The use of the terms “a”, “an”, and “the” in the context of describing the invention (especially in the context of the claims recited herein) are to be construed to cover both the singular and the plural, unless otherwise indicated herein or clearly contradicted by context. Furthermore, unless otherwise required by context, singular terms shall include pluralities and plural terms shall include the singular. Generally, nomenclatures used in connection with, and standard techniques described herein are those well known and commonly used in the art. Unless stated to the contrary, any use of the words such as “including,” “containing,” “comprising,” “having” and the like, means “including without limitation” and shall not be construed to limit any general statement that it follows to the specific or similar items or matters immediately following it.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention, thus generally described, will be understood more readily by reference to the following examples, which are provided by way of illustration and are not intended to be limiting of the present invention.

### EXAMPLE 1

### Dissolution Study

An exemplary oral solid dosage formulation of the present invention for combined administration of the compound of Formula II and Atazanavir was prepared as follows:

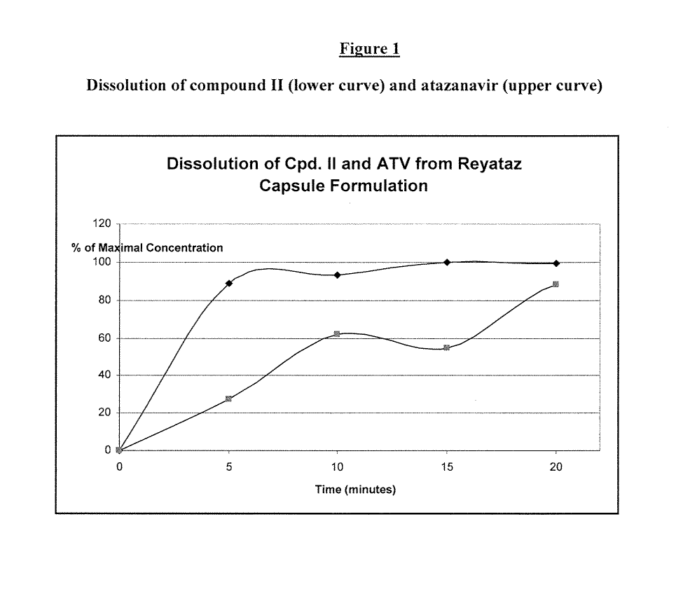

Commercially available 200 mg Reyataz hard gelatin capsules (lot number 6E3004B) were emptied by hand to create a stockpile of Atazanavir (ATV) commercial powder. This commercially available 200 mg Reyataz capsule contains 200 mg ATV as Atazanavir sulphate plus 178 mg of excipients (crospovidone, lactose monohydrate and magnesium stearate) for a total weigh to of 378 mg. 189 mg of the stockpiled commercial powder (equivalent to 100 mg Atazanavir) was blended by tumbling in a glass vial with 100 mg of the spray-dried amorphous dispersion (SDD) of Compound II/EUDRAGIT L-100 (EL-100) where the Compound II:EL100 ratio was 1:1 (produced by Hovione FarmaCientia) and filled by hand into “0” hard gelatin capsules. Therefore, each hand-filled “0” hard gelatin capsule contained 100 mg ATV, 50 mg of Compound II:EL100 (1:1) SDD and 89 mg of excipients. These capsules were dropped into 500 mL of simulated gastric fluid and the amounts of Compound II and ATV in the resulting suspension was determined by HPLC (see Table 1 and FIG. 1).

TABLE 1

| | | % of maximal |
|---|---|---|
| Time (min.) | % of maximal ATV Conc. | Compound II Conc. |
| 0 | 0 | 0 |
| 5 | 88.9 | 27.6 |
| 10 | 93.4 | 61.9 |
| 15 | 100.0 | 54.5 |
| 20 | 99.4 | 88.2 |
| 25 | 99.1 | 89.8 |
| 30 | 97.9 | 100.0 |

### EXAMPLE 2

### Pharmacokinetic Study

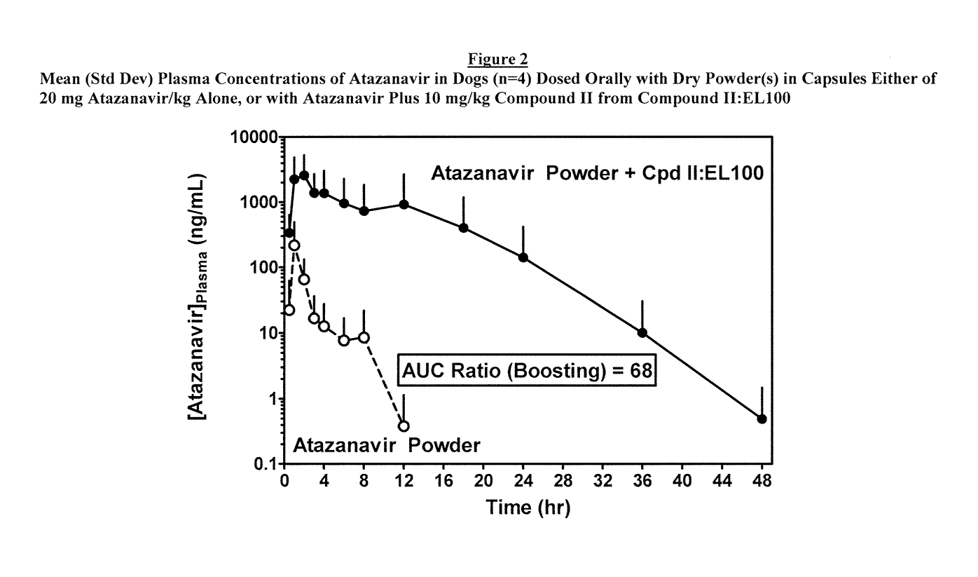

A pharmacokinetic study was performed in beagle dogs to evaluate the performance of the oral dosage formulation prepared in Example 1 above. The plasma exposure of Atazanavir from dry commercial powder formulation was markedly increased by co-administration with Compound II:EL100 (1:1) SDD powder (FIG. 2). The AUC of the men plasma Atazanavir concentrations was increased by a factor of 68. Both plasma levels and duration of exposure was increased. This demonstrated that Compound II:EL100 (1:1) SDD as a simple mixture of dry powder with Atazanavir in capsules was able to deliver the compound of Formula II effectively and generate the intended pharmacokinetic-enhancing effect on Atazanavir exposure.

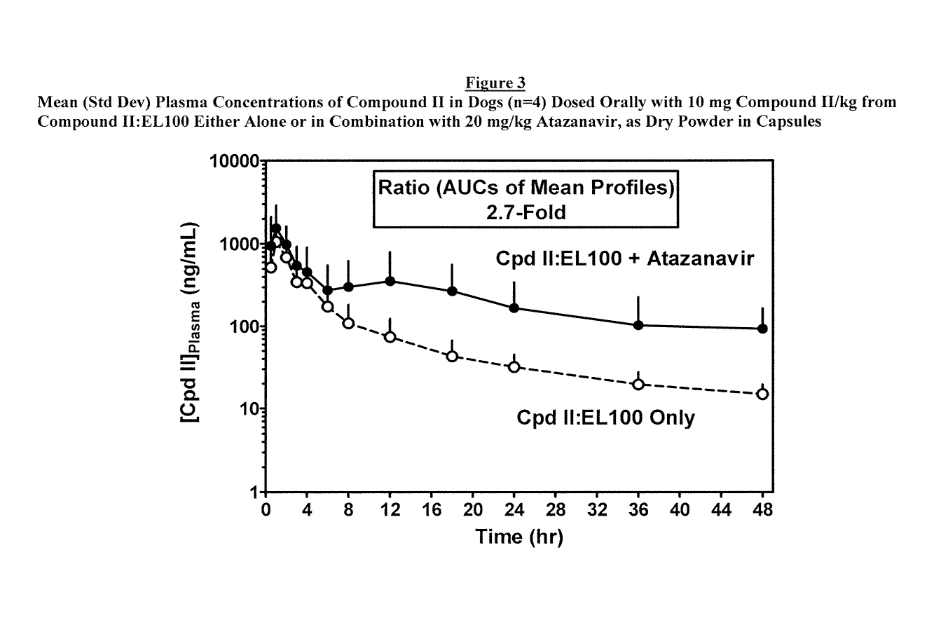

The plasma exposure of the compound of Formula II from Compound II:EL100 (1:1) SDD powder alone was compared to co-administration with Atazanavir powder (FIG. 3). The ratio of the AUCs from mean plasma concentrations was 2.5, but the differences among individual animal was not statistically significant. The apparent increase in mean Compound II exposure came mostly later in the time course, after 8 hours. The Cₘₐₓ was slightly increased and the plasma profiles were similar over about the first 6 hours. This demonstrated that co-administration of Atazanavir with Compound II:EL100 (1:1) SDD did not have a deleterious effect on Compound II plasma exposure. However, these results suggest that Atazanavir may have had an effect on the clearance of Compound II that was manifest at later time points.

Although the foregoing invention has been described in some detail by way of illustration and examples for purposes of clarity of understanding, it will be readily apparent to those of ordinary skill in the art in light of the teachings of this invention that certain variations and modifications may be made thereto without departing from the spirit or scope of the disclosure herein, including the specific embodiments. Thus, it is understood that modification and variation of the concepts herein disclosed may be resorted to by the skilled artisan, and that such modifications and variations are considered to be within the scope of this invention as defined by the appended claims.

What is claimed is:

1. A pharmaceutical composition comprising:
(i) an amorphous dispersion of an effective amount of a cytochrome p450 inhibitor and a water soluble polymer, wherein said amorphous dispersion has a glass transition temperature (Tg) of about 75° C. or greater and inhibits plasticization upon exposure to gastric fluid,
(ii) a disintegrant.

2. The composition of claim 1, further comprising an effective amount of an active pharmaceutical agent, wherein said active pharmaceutical agent is a substrate for human cytochrome p450.

3. The composition of claim 1, wherein said amorphous dispersion further comprises an effective amount of an active pharmaceutical agent, wherein said active pharmaceutical agent is a substrate for human cytochrome p450.

4. The composition of claim 1, wherein said cytochrome p450 inhibitor has the formula: 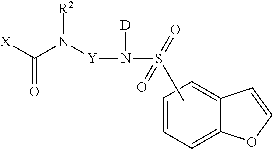
wherein:
X is C₁-C₁₂ alkyl, cycloalkyl, aryl, aralkyl, heteroaryl, or heteroaralkyl, optionally substituted with one or more substituents selected from the group consisting of halo, OR, ROH, R-halo, CN, COₙR, CON(R)₂, SOₙN(R)₂, SR, SOₙR, N(R)₂, N(R)COₙR, NRS(O)ₙR, oxo, and ═N—OR
Y is —(CG₁G₂)ₘ-, wherein m is 2-6 and wherein G₁ and G₂ are the same or different and wherein each G₁ and G₂ independently is selected from the group consisting of a bond, H, OR, optionally substituted alkyl, optionally substituted aryl, optionally substituted cycloalkyl, optionally substituted cycloalkylalkyl, optionally substituted aralkyl, optionally substituted heteroaryl, and optionally substituted heteroaralkyl, wherein each optional substitution independently is selected from the group consisting of alkyl , halo, cyano, CF₃, OR, C₃-C₇ cycloalkyl, C₅-C₇ cycloalkenyl, R6, OR2, SR2, N(R2)₂, OR3, SR3, NR2R3, OR6, SR6, and NR2R6,
D is selected from the group consisting of hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, O-cycloalkyl, O-cycloalkylalkyl, O-heterocycloalkyl, O-heterocycloalkylalkyl, O-heteroaralkyl O-aralkyl, N(R2)-alkyl, N(R2)-cycloalkyl, N(R2)-cycloalkylalkyl, N(R2)-heterocycloalkyl, N(R2)-heterocycloalkylalkyl, N(R2)-heteroaralkyl, N(R2)-aralkyl, wherein D optionally is substituted by alkyl, halo, nitro, cyano, O-alkyl, or S-alkyl;
wherein R is H, alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;
wherein each R2 is independently selected from the group consisting of H, C₁-C₁₂ alkyl, C₃-C₈ cycloalkyl, aryl, aralkyl, heteroaryl, heteroaralkyl, and heterocycloalkyl each further optionally substituted with one or more substituents selected from the group consisting of C₂-C₆ alkenyl, C₂-C₆ alkynyl, C₃-C₈ cycloalkyl, C₅-C8 cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);
or each R2 is independently selected from the group consisting of C₁-C₆ alkyl; substituted by aryl or heteroaryl; which groups optionally are substituted with one or more substituents selected from the group consisting of halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR;
R3 is C₂-C₆ alkenyl, C₂-C₆ alkynyl, C₃-C₈ cycloalkyl, C₅-C₈ cycloalkenyl, or heterocyclo; which groups optionally are substituted with one or more substituents selected from the group consisting of halo, OR2, R2-OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, oxo, ═N—OR2, ═N—N(R2)₂, ═NR2, ═NNRC(O)N(R2)₂, ═NNR2C(O)ₙR2, ═NNR2S(O)ₙN(R2)₂, and ═NNR2S(O)ₙ(R2);
R6 is aryl or heteroaryl, wherein said aryl or heteroaryl optionally are substituted with one or more groups selected from the group consisting of aryl, heteroaryl, R2, R3, halo, OR2, R2OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, OC(O)R2, OC(S)R2, OC(O)N(R2)₂, and OC(S)N(R2)₂; and
wherein n=1-2.

5. The composition of claim 4, wherein X is C₁-C₁₂ alkyl, m is 3, one G₁ is alkoxy, and a second G₁ is optionally substituted aralkyl, and D is alkyl.

6. The composition of claim 5, wherein said cytochrome p450 inhibitor has the formula:

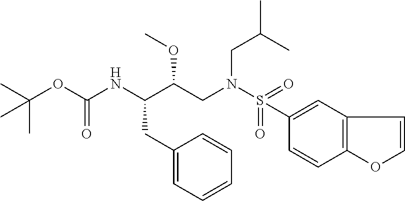

7. The composition of claim 1, wherein said water soluble polymer is selected from the group consisting of polyvinyl acetate phthalate, hydroxypropylmethyl-cellulose acetate succinate, cellulose acetate phthalate, methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate, cellulose propionate phthalate, cellulose acetate maleate, cellulose acetate trimellitate, cellulose acetate butyrate, cellulose acetate propionate, methacrylic acid/methacrylate polymer, methacrylic acid-methyl methacrylate copolymer, ethyl methacrylate-methylmethacrylate-chlorotrimethylammonium ethyl methacrylate copolymer, shellac, copal collophorium, carageenan, alginic acid and salts thereof, karaya gum, acacia gum, tragacanth gum, locust bean gum, guar gum, sodium carboxymethyl cellulose, methyl cellulose, and combinations thereof.

8. The composition of claim 7 wherein said water soluble polymer is a polymethacrylate.

9. (canceled)

10. The composition of claim 1, wherein said amorphous dispersion is a spray-dried dispersion, wherein said spray-dried dispersion comprises particles whose average diameter is <100 micron.

11. (canceled)

12. The composition of claim 1, wherein said dispersion has a glass transition temperature (Tg) between about 100° C. and about 125° C.

13. The composition of claim 2, wherein said active pharmaceutical agent is selected from the group consisting of Cyclosporine, Tacrolimus (FK506), Sirolimus (rapamycin), Indinavir, Ritonavir, Saquinavir, Felodipine, Isradipine, Nicardipine, Nisoldipine, Nimodipine, Nitrendipine, Nifedipine, Verapamil, Etoposide, Tamoxifen, Vinblastine, Vincristine, Taxol, Atorvastatin, Fluvastatin, Lovastatin, Pravastatin, Simvastatin, Terfenadine, Loratadine, Astemizole, Alfentanil, Carbamazepine, Azithromycin, Clarithromycin, Erythromycin, Itraconazole, Rifabutin, Lidocaine, Cisapride, Sertraline, Pimozide, Triazolam, Anastrazole, Busulfan, Corticosteroids (dexamethasone, methylprednisone and prednisone), Cyclophosphamide, Cytarabine, Docetaxel, Doxorubicin, Erlotinib, Exemestane, Gefitinib, Idarubicin, Ifosphamide, Imatinib mesylate, Irinotecan, Ketoconazole, Letrozole, Paclitaxel, Teniposide, Tretinoin, Vinorelbine,telithromycin: quinidine; alprazolam, diazepam, midazolam, nelfinavir, chlorpheniramine, amlodipine, diltiazem, lercanidipine, cerivastatin, estradiol, hydrocortisone, progesterone, testosterone, alfentanyl, aripiprazole, buspirone, cafergot, caffeine, cilostazol, codeine, dapsone, dextromethorphan, docetaxel, domperidone, eplerenone, fentanyl, finasteride, Gleevec®, haloperidol, irinotecan, Levo-Alpha Acetyl Methadol (LAAM), methadone, nateglinide, odanestron, propranolol, quinine, salmetrol, sildenafil, terfenadine, trazodone, vincristine, zaleplon, zolpidem., ixabepilone, Agenerase, Aptivus, Crixivan, Invirase, Lexiva, Prezista, Reyataz, Viracept, Elvitegravir, Selzentry, Vicriviroc, Telaprevir, Telithromycin, tandospirone, buspirone, pharmaceutically acceptable salts, crystalline forms, non-crystalline forms and polymorphs thereof.

14. (canceled)

15. The composition of claim 1, wherein said cytochrome p450 inhibitor and a water soluble polymer are present in a ratio ranging from 1.6:0.4 to 0.4:1.6.

16-18. (canceled)

19. The composition of claim 1, wherein the disintegrant is selected from the group consisting of microcrystalline cellulose, sodium starch glycolate, cross-linked carboxymethylcellulose and its sodium salt, cross-linked polyvinylpyrrolidone, pregelatinised starch, sodium carboxymethyl cellulose, calcium carboxymethyl cellulose, low-substituted hydroxypropyl cellulose, alginates or its salts and mixtures thereof.

20-24. (canceled)

25. The composition of claim 2, wherein said active pharmaceutical agent is atazanavir or atazanavir sulfate.

26-27. (canceled)

28. A method of inhibiting cytochrome p450 in a subject, comprising administering to said subject an effective amount of a composition according to claim 1.

29. A method of treating a patient suffering from HIV infection, comprising administering to said patient a composition according to claim 2, wherein said active pharmaceutical agent is an HIV inhibitor.

30. A method according to claim 29, wherein said HIV inhibitor is an HIV protease inhibitor.

31. A method of treating a patient suffering from HIV infection, comprising administering to said patient a composition according to claim 25.

32. A water-dispersible pharmaceutical dosage formulation suitable for oral administration comprising (i) an effective amount of a spray-dried amorphous dispersion of a compound having the formula: 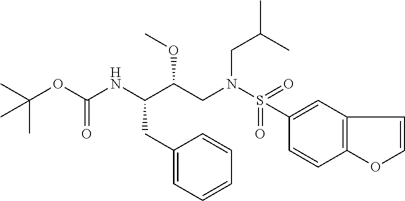
and a methacrylic acid-acrylic acid ethyl ester copolymer, wherein said dispersion has a glass transition temperature (Tg) in excess of 75° C., in combination with (ii) a drug selected from the group consisting of Cyclosporine, Tacrolimus (FK506), Sirolimus (rapamycin), Indinavir, Ritonavir, Saquinavir, Felodipine, Isradipine, Nicardipine, Nisoldipine, Nimodipine, Nitrendipine, Nifedipine, Verapamil, Etoposide, Tamoxifen, Vinblastine, Vincristine, Taxol, Atorvastatin, Fluvastatin, Lovastatin, Pravastatin, Simvastatin, Terfenadine, Loratadine, Astemizole, Alfentanil, Carbamazepine, Azithromycin, Clarithromycin, Erythromycin, Itraconazole, Rifabutin, Lidocaine, Cisapride, Sertraline, Pimozide, Triazolam, Anastrazole, Busulfan, Corticosteroids (dexamethasone, methylprednisone and prednisone), Cyclophosphamide, Cytarabine, Docetaxel, Doxorubicin, Erlotinib, Exemestane, Gefitinib, Idarubicin, Ifosphamide, Imatinib mesylate, Irinotecan, Ketoconazole, Letrozole, Paclitaxel, Teniposide, Tretinoin, Vinorelbine,telithromycin: quinidine; alprazolam, diazepam, midazolam, nelfinavir, chlorpheniramine, amlodipine, diltiazem, lercanidipine, cerivastatin, estradiol, hydrocortisone, progesterone, testosterone, alfentanyl, aripiprazole, buspirone, cafergot, caffeine, cilostazol, codeine, dapsone, dextromethorphan, docetaxel, domperidone, eplerenone, fentanyl, finasteride, gleevec, haloperidol, irinotecan, Levo-Alpha Acetyl Methadol (LAAM), methadone, nateglinide, odanestron, propranolol, quinine, salmetrol, sildenafil, terfenadine, trazodone, vincristine, zaleplon, zolpidem., ixabepilone, Agenerase, Aptivus, Crixivan, Invirase, Lexiva, Prezista, Reyataz,Viracept, Elvitegravir, Selzentry, Vicriviroc, Telaprevir, Telithromycin, tandospirone, buspirone, pharmaceutically acceptable salts, crystalline forms, non-crystalline forms and polymorphs thereof.

33. An oral solid gelatin capsule comprising:
(i) an effective amount of a spray-dried amorphous dispersion of a compound having the formula: 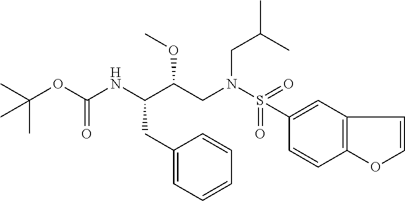
and a methacrylic acid-acrylic acid ethyl ester copolymer in the ratio 1:1, wherein said dispersion has a glass transition temperature (Tg) in excess of 75° C.;
(ii) atazanavir sulphate in a powder form wherein said powder comprises crospovidone, lactose monohydrate and magnesium stearate.
